(19) United States
(12) Patent Application Publication
(10) Pub. No.: US 2019/0142506 A1
(43) Pub. Date: May 16, 2019

(54) ENERGY TREATMENT INSTRUMENT, CONTROL DEVICE, AND TREATMENT SYSTEM

(71) Applicant: OLYMPUS CORPORATION, Tokyo (JP)

(72) Inventors: Hiroshi MIYAJIMA, Hachioji-shi (JP); Kazue TANAKA, Sagamihara-shi (JP)

(73) Assignee: OLYMPUS CORPORATION, Tokyo (JP)

(21) Appl. No.: 16/248,163

(22) Filed: Jan. 15, 2019

Related U.S. Application Data
(63) Continuation of application No. PCT/JP2016/070973, filed on Jul. 15, 2016.

(51) Int. Cl. A61B 18/14; A61B 17/32

(52) U.S. Cl. CPC A61B 18/1447; A61B 2018/00607; A61B 17/320092; A61B 18/1445

(57) ABSTRACT

An end effector grasps a treatment target between a pair of gasping pieces, and an energy output source outputs electric energy so that treatment energy is applied to the grasped treatment target. A processor switches a driving state of an actuator based on a fact that a state of the treatment target has become a predetermined state when the electric energy is output from the energy output source. As the result, a state of the end effector is switched from a first grasping state to a second grasping state, a grasping force and a grasping pressure between the first grasping piece and the second grasping piece in the second grasping state being different from those in the first grasping state.

### CROSS-REFERENCE TO RELATED APPLICATIONS

This is a Continuation Application of PCT Application No. PCT/JP2016/070973, filed Jul. 15, 2016, the entire contents of which are incorporated herein by reference.

### BACKGROUND OF THE INVENTION

### Field of the Invention

The present invention relates to an energy treatment instrument in which a treatment target such as a biological tissue or the like is grasped between a pair of grasping pieces and treated by using treatment energy such as an ultrasonic vibration, a high-frequency current, and the like. Further, the present invention relates to a control device used together with the energy treatment instrument, and a treatment system including the energy treatment instrument.

### Description of the Related Art

A specification of U.S. Patent Application Publication No. 2009/0270853 discloses an energy treatment instrument in which a pair of grasping pieces is provided in an end effector and a treatment target such as a biological tissue or the like can be grasped between the pair of grasping pieces. In the energy treatment instrument, each of the grasping pieces includes an electrode. Further, as electric energy is supplied to the electrodes of both grasping pieces, a high-frequency current flows between the electrodes through the grasped treatment target. The treatment target is coagulated as the high-frequency current is applied as treatment energy to the treatment target. In addition, an ultrasonic transducer is provided in the energy treatment instrument and electric energy is supplied to the ultrasonic transducer, such that an ultrasonic vibration is generated. The generated ultrasonic vibration is transmitted to one of the grasping pieces, and applied to the grasped treatment target. The treatment target is cut open as the ultrasonic vibration is applied as treatment energy to the treatment target.

### BRIEF SUMMARY OF THE INVENTION

According to one aspect of the invention, an energy treatment instrument used with a control device, the control device including a processor, the energy treatment instrument including: an end effector which includes a first grasping piece, and a second grasping piece being openable and closable relative to the first grasping piece, the end effector being configured to grasp a treatment target between the first grasping piece and the second grasping piece, and treatment energy being configured to be applied to the grasped treatment target; an actuator which is configured to be driven in accordance with a control by the processor, a driving state of the actuator being configured to be switched based on a fact that a state of the treatment target has become a predetermined state when the treatment energy is applied to the grasped treatment target, so that a state of the end effector is switched from a first grasping state to a second grasping state, and the actuator being configured to make a grasping force and a grasping pressure between the first grasping piece and the second grasping piece in the second grasping state different from those in the first grasping state; a housing which is hand-holdable; and a handle which is configured to open or close relative to the housing so as to open or close the first grasping piece and the second grasping piece relative to each other, wherein a stroke of the handle in a closing motion is configured to be changed in accordance with a switch of the driving state of the actuator, so that the state of the end effector is switched from the first grasping state to the second grasping state.

According to one another aspect of the invention, a control device used with an energy treatment instrument and an actuator, the energy treatment instrument including a first grasping piece, a second grasping piece, a hand-holdable housing, and a handle, the second grasping piece being openable and closable relative to the first grasping piece, the handle being configured to open or close relative to the housing so as to open or close the first grasping piece and the second grasping piece relative to each other, an end effector configured to grasp a treatment target between the first grasping piece and the second grasping piece being provide in the energy treatment instrument, the control device including: an energy output source configured to output electric energy, the energy output source being configured to supply the output electric energy to the energy treatment instrument so that treatment energy is applied to the grasped treatment target through the end effector; and a processor which is configured to control an output of the electric energy from the energy output source and control a driving of the actuator, the processor being configured to switch a driving state of the actuator based on a fact that a state of the treatment target has become a predetermined state when the electric energy is output from the energy output source, so that a stroke of the handle in a closing motion is changed, a state of the end effector being configured to be switched from a first grasping state to a second grasping state in accordance with a change of the stroke of the handle, the actuator being configured to make a grasping force and a grasping pressure between the first grasping piece and the second grasping piece in the second grasping state different from those in the first grasping state.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing a treatment system according to a first embodiment,

FIG. 2 is a block diagram showing a configuration related to a control of an energy treatment instrument by a control device according to the first embodiment,

FIG. 3 is a schematic diagram showing a configuration for changing a grasping force and a grasping pressure between grasping pieces in one example according to the first embodiment,

FIG. 4 is a schematic diagram showing a configuration for changing a grasping force and a grasping pressure between grasping pieces in another example according to the first embodiment,

FIG. 5 is a flowchart showing a process executed by a processor according to the first embodiment,

FIG. 6A is a schematic diagram showing an example of a change with time in impedance of a treatment target in a state where treatment energy is applied to the treatment target,

FIG. 6B is a schematic diagram showing a change with time in a grasping force between the grasping pieces in the case where impedance is changed as shown in FIG. 6A in the first embodiment,

FIG. 6C is a schematic diagram showing switching with time between an ON state and an OFF state of an output of electric energy to an ultrasonic transducer in the case where impedance is changed as shown in FIG. 6A in the first embodiment,

FIG. 7 is a schematic diagram showing a change with time in a grasping force between the grasping pieces in the case where impedance is changed as shown in FIG. 6A in one modification of the first embodiment, and

FIG. 8 is a schematic diagram showing a change with time in a grasping force between the grasping pieces in the case where impedance is changed as shown in FIG. 6A in another modification of the first embodiment.

### DETAILED DESCRIPTION OF THE INVENTION

### First Embodiment

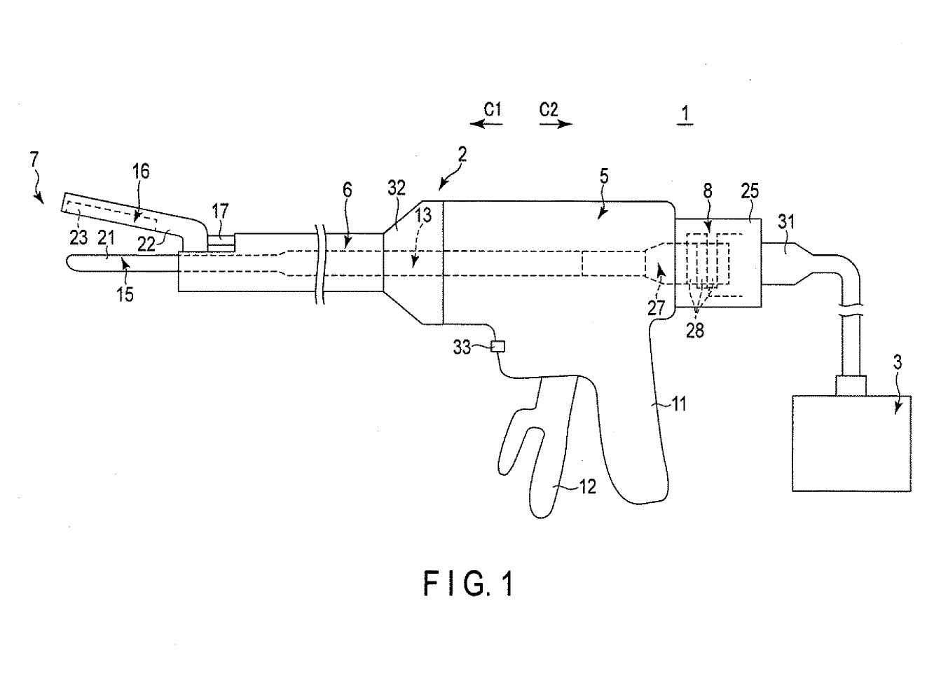

An embodiment and the like of the present invention will be described with reference to FIGS. 1 to 6C. FIG. 1 is a diagram showing a treatment system 1. As shown in FIG. 1, the treatment system 1 includes an energy treatment instrument 2 and a control device 3. The energy treatment instrument 2 has a longitudinal axis C. Here, one side in a direction along the longitudinal axis C is a distal side (arrow C1 side), and the other side opposite to the distal side is a proximal side (arrow C2 side).

The energy treatment instrument 2 includes a housing 5 which can be hand-held, a shaft (sheath) 6 connected to a distal side of the housing 5, an end effector 7 provided at a distal portion of the shaft 6, and a transducer unit 8 connected to the housing 5 from the proximal side. A central axis of the shaft 6 is approximately coaxial with the longitudinal axis C. The housing 5 is provided with a grip 11, and a handle 12 is pivotably attached to the housing 5. As the handle 12 pivots relative to the housing 5, the handle 12 is opened or closed relative to the grip 11. In the present embodiment, the handle 12 is provided on the distal side with respect to the grip 11, and a movement direction of an opening motion and a closing motion of the handle 12 is approximately parallel to the longitudinal axis C, but it is not limited thereto. In one example, the handle 12 is provided on the proximal side with respect to the grip 11. In another example, the handle 12 is provided on a side opposite to the grip 11 with respect to the longitudinal axis, and a movement direction of the opening motion and the closing motion of the handle 12 is approximately perpendicular to the longitudinal axis C.

In the energy treatment instrument 2, a rod member (probe) 13 extends from the inside of the housing 5 toward the distal side through the inside of the shaft 6. The rod member 13 is formed of a material with high vibration transmission property, such as 64 titanium (Ti-6Al-4V) or the like. A first grasping piece (treatment section) 15 is provided at a distal portion of the rod member 13. The rod member 13 is inserted through the shaft 6 in a state where the first grasping piece 15 protrudes from a distal end of the shaft 6 toward the distal side.

Further, a second grasping piece (jaw) 16 is provided at the distal portion of the shaft 6. A movable member 17 extends inside the shaft 6 from the proximal side toward the distal side. A distal portion of the movable member 17 is connected to the second grasping piece 16, and a proximal portion of the movable member 17 is connected to the handle 12 inside the housing 5. The movable member 17 moves toward the proximal side or the distal side by opening or closing the handle 12 relative to the grip 11. By doing so, the second grasping piece 16 pivots around the shaft 6, and is opened or closed relative to the first grasping piece 15. That is, a pair of grasping pieces 15 and 16 can be opened and closed relative to each other. In the present embodiment, the end effector 7 includes the pair of grasping pieces 15 and 16. In the end effector 7, the treatment target such as a biological tissue (blood vessel) or the like is grasped between the grasping pieces 15 and 16 by closing the pair of grasping pieces 15 and 16 relative to each other.

The first grasping piece 15 includes a first electrode 21 formed of a conductive material. Further, the second grasping piece 16 includes a second electrode 22 formed of a conductive material, and a pad member 23 attached to the second electrode 22. The pad member 23 is formed of a resin such as a polytetrafluoroethylene (PTFE) or the like, and formed of an electrically insulating material. In a state where the grasping pieces 15 and 16 are closed relative to each other, the pad member 23 can abut the first grasping piece 15. In a state where the pad member 23 abuts the first grasping piece 15, the second electrode 22 is not in contact with the first grasping piece 15 (first electrode 21).

The transducer unit 8 includes a transducer case 25, and an ultrasonic transducer 27 provided inside the transducer case 25. The ultrasonic transducer 27 is connected to a proximal side of the rod member 13 inside the housing 5. The ultrasonic transducer 27 includes at least one piezoelectric element 28. One end of a cable 31 is connected to the transducer case 25. The other end of the cable 31 is detachably connected to the control device 3. In one example, the transducer case 25 is not provided and the ultrasonic transducer 27 is disposed inside the housing 5. In this case, one end of the cable 31 is connected to the housing 5.

Further, in the present embodiment, a rotation member (rotary knob) 32 is attached to the housing 5. By rotating the rotation member 32, the shaft 6, the rod member 13 including the first grasping piece 15, the second grasping piece 16, and the ultrasonic transducer 27 rotate together with the rotation member 32 around the longitudinal axis C relative to the housing 5. In this manner, an angular position of the end effector 7 around the longitudinal axis C is adjusted. In one example, the rotation member 32 may not be provided.

In addition, an operation button 33 is installed in the housing 5. An operation for supplying electric energy from the control device 3 to the energy treatment instrument 2 is input through the operation button 33. That is, a state of the operation for supplying electric energy from the control device 3 to the energy treatment instrument 2 can be switched between an ON state and an OFF state by using the operation button 33. In one example, a foot switch may be provided separately from the energy treatment instrument 2 in place of the operation button 33, or in addition to the operation button 33.

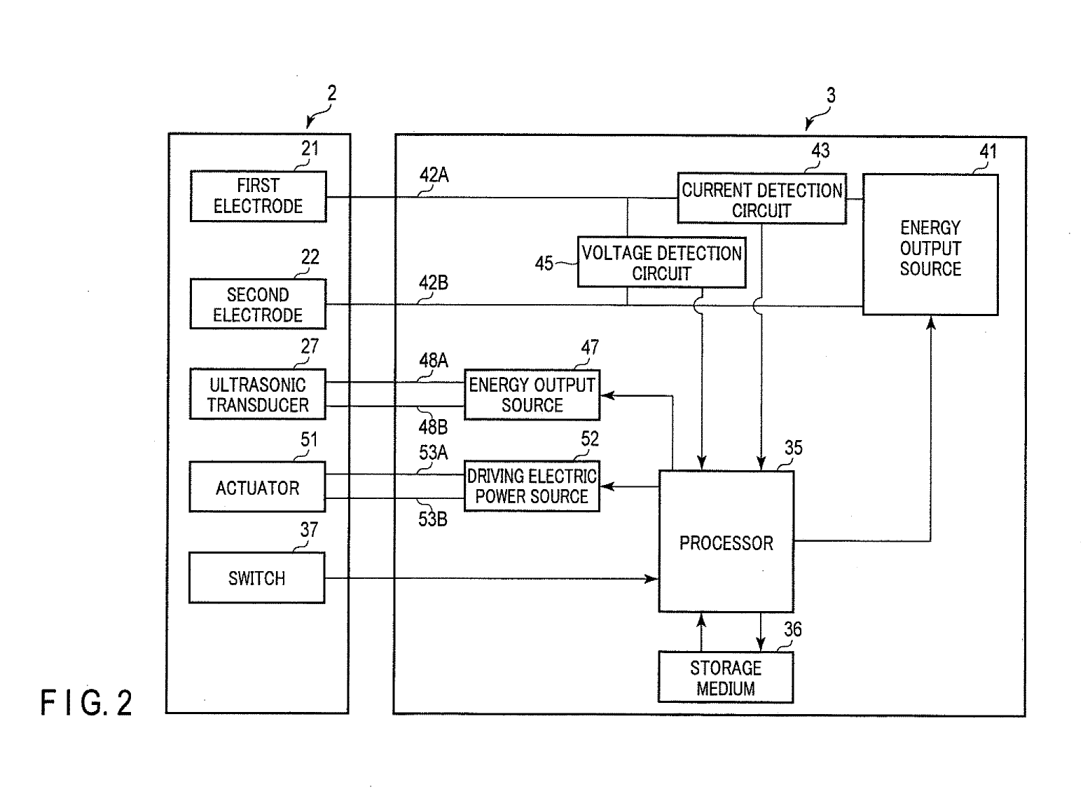

FIG. 2 is a diagram showing a configuration related to a control of the energy treatment instrument 2 by the control device 3. As shown in FIG. 2, the control device 3 includes a processor 35 controlling the entire treatment system 1, and a storage medium 36. The processor (controller) 35 is formed of an integrated circuit including a central processing unit (CPU), a digital signal processor (DSP), an application specific integrated circuit (ASIC), a field programmable gate array (FPGA), or the like. The processor 35 may be formed of a plurality of integrated circuits, and may be formed of one integrated circuit. Further, one processor 35 may be provided in the control device 3, and a plurality of processors 35 may be separately provided in the control device 3. The process executed by the processor 35 is executed in accordance with a program stored in the processor 35 or the storage medium 36. Further, the storage medium 36 stores a processing program for use in the processor 35 and parameters, tables, and the like for use in an arithmetic process executed by the processor 35.

A switch 37 is provided inside the housing 5 of the energy treatment instrument 2. As an operation input is made through the operation button 33, a state of the switch 37 is switched from an OFF state to an ON state. The processor 35 detects an input of an operation through the operation button 33 based on switching of the state of the switch 37 to the ON state. Further, the processor 35 detects that the operation input is continuously made through the operation button 33 based on the fact that the ON state of the switch 37 is maintained.

The control device 3 includes an energy output source (HF electric power source) 41. The energy output source 41 is electrically connected to the first electrode 21 of the first grasping piece 15 via an electricity path 42A that extends inside the cable 31 and inside the housing 5. The energy output source 41 is electrically connected to the second electrode 22 of the second grasping piece 16 via an electricity path 42B that extends inside the cable 31 and inside the housing 5. The energy output source 41 includes a conversion circuit or the like which converts electric power from a battery power source or a power receptacle into electric energy supplied to the electrodes 21 and 22. The energy output source 41 outputs the electric energy converted in the conversion circuit. Then, the electric energy output from the energy output source 41 is supplied to the electrodes 21 and 22 via the electricity paths 42A and 42B. The processor 35 controls an output of the electric energy from the energy output source 41. It should be noted that high-frequency electric power is output as electric energy from the energy output source 41.

As electric energy is supplied to the electrodes 21 and 22, a high-frequency current flows between the electrodes 21 and 22 through the treatment target grasped between the grasping pieces 15 and 16. By doing so, the high-frequency current is applied as first treatment energy to the grasped treatment target.

The control device 3 includes a current detection circuit 43 and a voltage detection circuit 45. The current detection circuit 43 detects an output current I from the energy output source 41 to the electrodes 21 and 22, and the voltage detection circuit 45 detects an output voltage V from the energy output source 41 to the electrodes 21 and 22. A detection result of the output current I obtained by the current detection circuit 43 and a detection result of the output voltage V obtained by the voltage detection circuit 45 are transferred to the processor 35. In one example, the processor 35 detects impedance Z of the grasped treatment target based on the detection results of the output current I and the output voltage V. Further, in one example, the processor 35 detects a phase difference θ between the output current I and the output voltage V based on the detection results of the output current I and the output voltage V. Further, in one example, the processor 35 detects a duration time T for which the output of the electric energy from the energy output source 41 is continued from the start of the output. The control device 3 includes an energy output source (US electric power source) 47 separately from the energy output source 41. The energy output source 47 is electrically connected to the ultrasonic transducer 27 via electricity paths 48A and 48B that extend inside the cable 31. The energy output source 47 includes a conversion circuit or the like which converts electric power from a battery power source or a power receptacle into electric energy supplied to the ultrasonic transducer 27. The energy output source 47 outputs electric energy converted in the conversion circuit. Then, the electric energy output from the energy output source 47 is supplied to the ultrasonic transducer 27 via the electricity paths 48A and 48B. The processor 35 controls an output of the electric energy from the energy output source 47. It should be noted that alternating current (AC) electric power is output as electric energy from the energy output source 47 at a certain frequency in a predetermined frequency range.

As the electric energy is supplied to the ultrasonic transducer 27, a voltage is applied to each piezoelectric element 28, and AC current flows to each piezoelectric element 28. As a result, the AC current is converted into an ultrasonic vibration by the piezoelectric element 28, such that the ultrasonic vibration is generated in the ultrasonic transducer 27. The generated ultrasonic vibration is transmitted from the proximal side to the distal side through the rod member 13. Further, the ultrasonic vibration is transmitted to the first grasping piece 15, such that the rod member 13 including the first grasping piece 15 vibrates. At this time, the rod member 13 vibrates approximately in parallel to the longitudinal direction of the rod member 13 at a certain frequency (for example, 47 kHz) in a predetermined frequency range (for example, 46 kHz to 48 kHz). The ultrasonic vibration transferred to the first grasping piece 15 is applied as second treatment energy to the grasped treatment target through the end effector 7.

In addition, an actuator 51 is provided in the energy treatment instrument 2. The actuator 51 is, for example, an electromagnetic solenoid, an electric motor, or the like, and is provided inside the housing 5. In addition, a driving electric power source 52 is provided in the control device 3. The driving electric power source 52 is electrically connected to the actuator 51 via electricity paths 53A and 53B that extend inside the cable 31. The driving electric power source 52 includes a conversion circuit or the like which converts electric power from a battery power source or a power receptacle into driving electric power of the actuator 51. The driving electric power source 52 outputs driving electric power converted in the conversion circuit. Then, the driving electric power output from the driving electric power source 52 is supplied to the actuator 51 via the electricity paths 53A and 53B. The processor 35 controls an output of the driving electric power from the driving electric power source 52. By doing so, the processor 35 controls a supply of the driving electric power to the actuator 51, thereby controlling the driving of the actuator 51. In one example, the actuator 51 may be provided separately from the energy treatment instrument 2.

As a driving state of the actuator 51 is changed in accordance with the control by the processor 35, a grasping force and a grasping pressure between the grasping pieces 15 and 16 are changed in the end effector 7. Here, the grasping force is a pressing force applied from the second grasping piece 16 to the first grasping piece 15 in a state where the treatment target is grasped. Further, the grasping pressure is a force to crush the treatment target grasped between the grasping pieces 15 and 16.

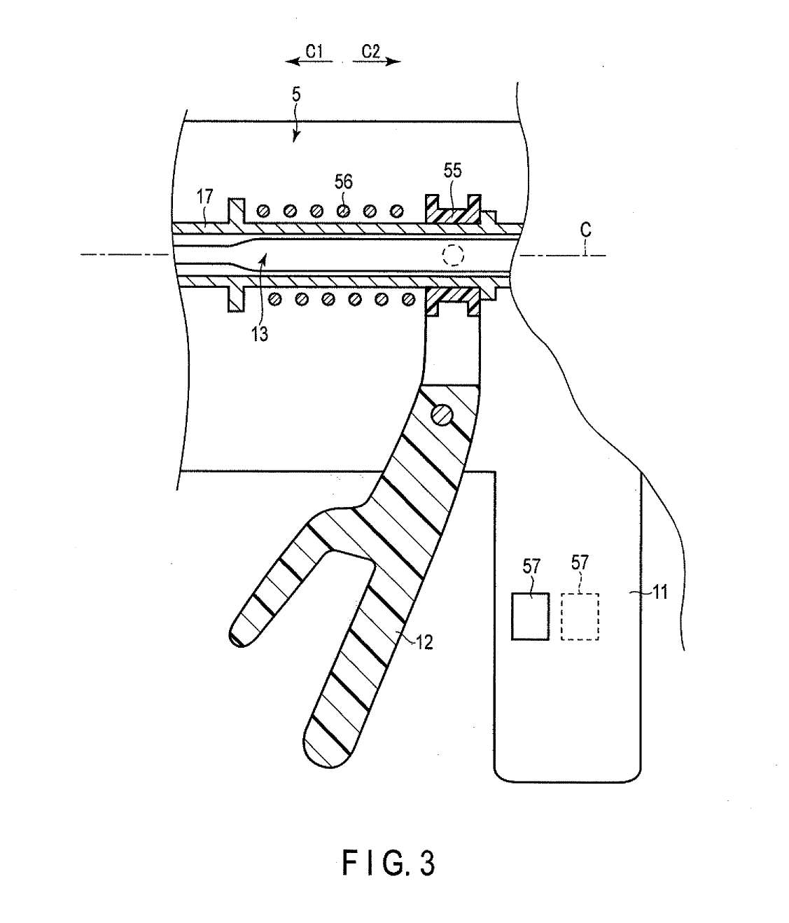

FIG. 3 is a diagram showing a configuration for changing a grasping force and a grasping pressure between the grasping pieces 15 and 16 in one example. As shown in FIG. 3, a proximal portion of the movable member 17 is inserted into the housing 5. In addition, a slider member 55 is disposed on an outer circumferential surface of the movable member 17 inside the housing 5. The slider member 55 can move along the longitudinal axis C relative to the movable member 17. The handle 12 is attached to the slider member 55. In addition, an elastic member 56 such as a coil spring, or the like is disposed on the outer circumferential surface of the movable member 17. A proximal end of the elastic member 56 is connected to the slider member 55, and a distal end of the elastic member 56 is connected to the movable member 17. In a state where the handle 12 is maximally opened relative to the grip 11, that is, in a state where the grasping pieces 15 and 16 are maximally opened relative to each other, the elastic member 56 is in a reference state where the elastic member 56 contracts from a natural state by a displacement amount x0. At this time, when an elastic modulus of the elastic member 56 is k0, an elastic force having a magnitude of k0x0 is applied from the elastic member 56 to the movable member 17.

When the handle 12 is closed relative to the grip 11 in the state where the handle 12 is maximally opened relative to the grip 11, the movable member 17 and the slider member 55 move together toward the distal side so as to close the grasping pieces 15 and 16 relative to each other until the treatment target grasped between the grasping pieces 15 and 16 is crushed to some extent. For this reason, the elastic member 56 cannot contract from the reference state until the grasped treatment target is crushed to some extent, and thereby the magnitude of the elastic force applied from the elastic member 56 to the movable member 17 is not changed from k0x0.

Then, when the grasped treatment target is crushed to some extent, the closing motion of the second grasping piece 16 is stopped, and movement of the movable member 17 toward the distal side is stopped. In this state, when the handle 12 is further closed relative to the grip 11, the slider member 55 moves to the distal side relative to the movable member 17. By doing so, the elastic member 56 further contacts from the reference state. Here, in a state where the elastic member 56 contracts by a displacement amount (contraction amount) x from the reference state, a magnitude of the elastic force applied from the elastic member 56 to the movable member 17 is k0 (x0+x), which is larger than that of the elastic force in the case of the reference state.

Here, when the displacement amount x of the elastic member 56 is increased and the elastic force applied from the elastic member 56 to the movable member 17 is increased, the grasping force and the grasping pressure between the grasping pieces 15 and 16 are increased. That is, as a contraction state of the elastic member 56 is changed, a grasping force and a grasping pressure between the grasping pieces 15 and 16 are changed.

In addition, a stopper member 57 is provided in the housing 5. As the handle 12 abuts the stopper member 57, a closing motion of the handle 12 relative to the grip 11 is restricted. That is, the handle 12 is closed relative to the grip 11 until the handle 12 abuts the stopper member 57.

The stopper member 57 can move between a first position (a position indicated by a broken line in FIG. 3) and a second position (a position indicated by a solid line in FIG. 3) in accordance with the driving state of the actuator 51. In one example, the actuator 51 is an electromagnetic solenoid, and, when driving electric power is not supplied to the actuator 51, the stopper member 57 is located in the second position by urging of an urging member (not shown) or the like. In addition, in a state where driving electric power is supplied to the actuator 51, the stopper member 57 moves from the second position to the first position by an electromagnetic force of the electromagnetic solenoid, resisting the urging.

In the state where the stopper member 57 is located in the second position indicated by a solid line, a stroke of the handle 12 in the closing motion is smaller than that in the state where the stopper member 57 is located in the first position indicated by a broken line. That is, as the driving state of the actuator 51 is switched in accordance with the control by the processor 35, the stroke of the handle 12 in the closing motion is changed.

In a state where the handle 12 abuts the stopper member 57 located in the first position, the end effector 7 is in a first grasping state. In a state where the handle 12 abuts the stopper member 57 located in the second position, the end effector 7 is in a second grasping state which is different from the first grasping state. As described above, in the state where the stopper member 57 is located in the second position, a stroke of the handle 12 in the closing motion is smaller than that in the state where the stopper member 57 is located in the first position. For this reason, a displacement amount x2 of the elastic member 56 in the second grasping state of the end effector 7 from the reference state is smaller than a displacement amount x1 of the elastic member 56 in the first grasping state of the end effector 7 from the reference state, and a magnitude k0 (x0+x2) of the elastic force applied from the elastic member 56 to the movable member 17 in the second grasping state is smaller than a magnitude k0 (x0'x1) of the elastic force applied from the elastic member 56 to the movable member 17 in the first grasping state. Accordingly, the grasping force and the grasping pressure between the grasping pieces 15 and 16 are smaller in the second grasping state than in the first grasping state.

In the present example as described above, as the driving state of the actuator 51 is switched in accordance with the control by the processor 35, the stroke of the handle 12 in the closing motion is changed and the contraction state of the elastic member 56 is changed. As a result, for example, the state of the end effector 7 is switched from the first grasping state to the second grasping state, and the grasping force and the grasping pressure between the grasping pieces 15 and 16 are changed.

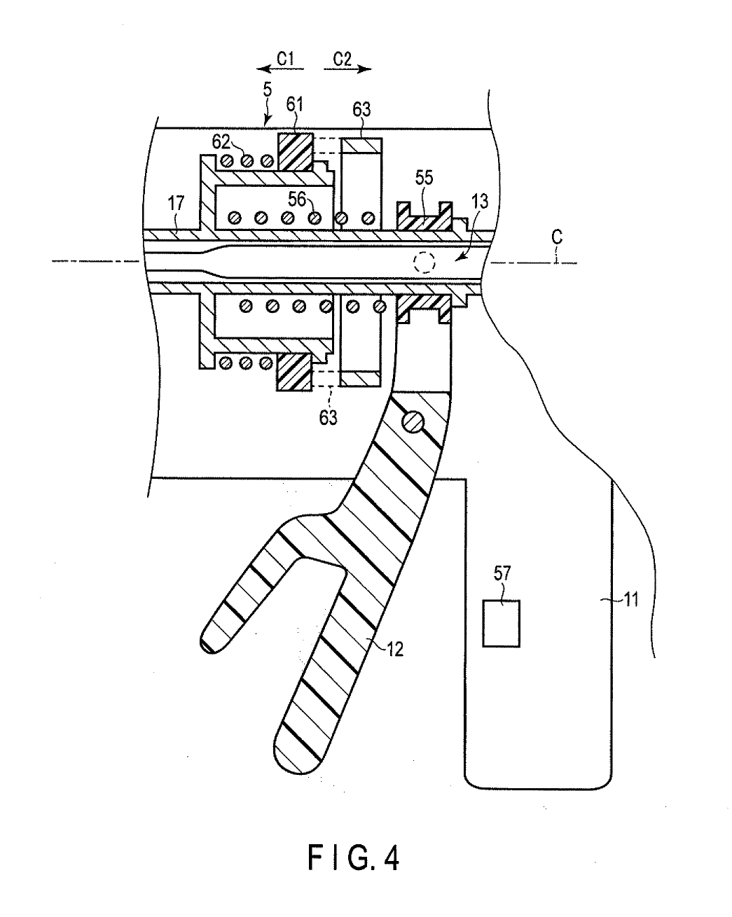

FIG. 4 is a diagram showing a configuration for changing a grasping force and a grasping pressure between the grasping pieces 15 and 16 in another example. As shown in FIG. 4, also in the present example, the slider member 55, the elastic member 56, and the stopper member 57 are provided as well, similarly to the example of FIG. 3. Also in the present example, when the handle 12 is closed, the movable member 17 and the slider member 55 move together toward the distal side and the grasping pieces 15 and 16 are closed relative to each other until the grasped treatment target is crushed to some extent. Then, in a state where the grasped treatment target is crushed to some extent, when the handle 12 is further closed relative to the grip 11, the slider member 55 moves toward the distal side relative to the movable member 17, and the elastic member 56 further contracts from the reference state.

However, in the present example, the stopper 57 does not move, but is fixed to the housing 5. In addition, in the present example, a slider member 61 and an elastic member 62 such as a coil spring or the like are attached to the movable member 17. The slider member 61 can move along the longitudinal axis C relative to the movable member 17. A proximal end of the elastic member 62 is connected to the slider member 61, and a distal end of the elastic member 62 is connected to the movable member 17.

Further, in the present example, a pressing member 63 which can press the slider member 61 from the proximal side is provided inside the housing 5. The pressing member 63 can move between a first position (a position indicated by a broken line in FIG. 4) and a second position (a position indicated by a solid line in FIG. 4) in accordance with the driving state of the actuator 51. In one example, the actuator 51 is an electromagnetic solenoid, and, when driving electric power is not supplied to the actuator 51, the pressing member 63 is located in the second position by urging of an urging member (not shown) or the like. In addition, in a state where driving electric power is supplied to the actuator 51, the pressing member 63 moves from the second position to the first position by an electromagnetic force of the electromagnetic solenoid, resisting the urging.

In a state where the pressing member 63 is located in the second position, the pressing member 63 is not in contact with the slider member 61. At this time, the elastic member 62 is in a reference state where the elastic member 62 contracts from a natural state by a displacement amount x′0. For this reason, when an elastic modulus of the elastic member 62 is k′0, an elastic force having a magnitude of k′0x′0 is applied from the elastic member 62 to the movable member 17. Meanwhile, in a state where the pressing member is located in the first position, the pressing member 63 presses the slider member 61 from the proximal side. As the slider member 61 is pressed by the pressing member 63, the elastic member 62 further contracts by a displacement amount (contraction amount) x′1 from the reference state. As a result, a magnitude of the elastic force applied from the elastic member 62 to the movable member 17 is k′0 (x′0+x′1), which is larger than that of the elastic force in the case of the reference state.

Here, in the present example, the grasping force and the grasping pressure between the grasping pieces 15 and 16 are changed depending on a contraction state of the elastic member 62 in addition to the contraction state of the elastic member 56. Accordingly, when the displacement amount x′ of the elastic member 62 from the reference state is increased and the elastic force applied from the elastic member 62 to the movable member 17 is increased, the grasping force and the grasping pressure between the grasping pieces 15 and 16 are increased.

In the present example, in a state where the handle 12 abuts the stopper member 57 and the pressing member 63 is located in the first position, the end effector 7 is in the first grasping state. Further, in a state where the handle 12 abuts the stopper member 57 and the pressing member 63 is located in the second position, the end effector 7 is in the second grasping state. The elastic force applied from the elastic member 56 to the movable member 17 in the first grasping state and the elastic force applied from the elastic member 56 to the movable member 17 in the second grasping state are approximately the same as each other. However, a magnitude k′0x′0 of the elastic force applied from the elastic member 62 to the movable member 17 in the second grasping state is smaller than a magnitude k′0 (x′0+x′1) of the elastic force applied from the elastic member 62 to the movable member 17 in the first grasping state of the end effector 7. Accordingly, the grasping force and the grasping pressure between the grasping pieces 15 and 16 are smaller in the second grasping state than in the first grasping state.

In the present example as described above, as the driving state of the actuator 51 is switched in accordance with the control by the processor 35, the contraction state of the elastic member 56 is changed. As a result, for example, the state of the end effector 7 is switched from the first grasping state to the second grasping state, and the grasping force and the grasping pressure between the grasping pieces 15 and 16 are changed.

It should be noted that the configuration for changing the grasping force and the grasping pressure between the grasping pieces 15 and 16 in accordance with the switching of the driving state of the actuator 51 is not limited to the example described above. For example, the grasping force and the grasping pressure between the grasping pieces 15 and 16 may be changed by applying the actuator 51 driven in accordance with the control by the processor 35 to the same structure as any structures disclosed in International Publication No. WO2013/141217 (PCT Application No. PCT/JP2013/057712). Also in this case, as the driving state of the actuator 51 is switched in accordance with the control by the processor 35, the grasping force and the grasping pressure between the grasping pieces 15 and 16 are changed.

Further, in one example, in a state where the driving electric power is not supplied to the actuator 51, the end effector 7 may be in the first grasping state, and in a state where the driving electric power is supplied to the actuator 51, the end effector 7 may be in the second grasping state where the grasping force and the grasping pressure are smaller than those in the first grasping state. Further, in another example, the driving electric power may be supplied to the actuator 51 in both of the first grasping state of the end effector 7 and the second grasping state where the grasping force and the grasping pressure are smaller than those in the first grasping state, and a direction of a driving current supplied to the actuator 51 in the first grasping state and a direction of a driving current supplied to the actuator 51 in the second grasping state may be opposite to each other. Further, in another example, the state of the end effector 7 may be switched to three or more grasping states with different grasping forces and different grasping pressures from one another depending on the control of the driving of the actuator 51 by the processor 35. In this case, the three or more grasping states include the first grasping state and the second grasping state where the grasping force and the grasping pressure are smaller than those in the first grasping state.

Next, a function and an effect of the present embodiment will be described. When performing the treatment using the treatment system 1, a surgeon holds the housing 5 and inserts the end effector 7 into a body cavity such as the abdominal cavity or the like in the body. Further, the treatment target such as the blood vessel or the like is put between the grasping pieces 15 and 16, and the handle 12 is closed relative to the grip 11. By doing so, the treatment target is grasped between the grasping pieces 15 and 16. In this state, an operation input is made by the surgeon through the operation button 33. By doing so, the grasped treatment target is treated by using treatment energy such as a high-frequency current, an ultrasonic vibration, and the like. Here, a high-frequency current flows to the grasped treatment target through the grasping pieces 15 and 16, and the treatment target is coagulated by heat caused by the high-frequency current. Further, as the first grasping piece 15 vibrates due to the ultrasonic vibration, the treatment target is cut open by frictional heat generated between the first grasping piece 15 and the treatment target. Accordingly, in the present embodiment, the high-frequency current is the first treatment energy coagulating the treatment target, and the ultrasonic vibration is the second treatment energy cutting the treatment target open. Further, in a state where the treatment energy is applied to the treatment target, the processor 35 controls the driving of the actuator 51 by controlling an output of driving electric power from the driving electric power source 52. By doing so, the grasping force and the grasping pressure between the grasping pieces 15 and 16 are adjusted.

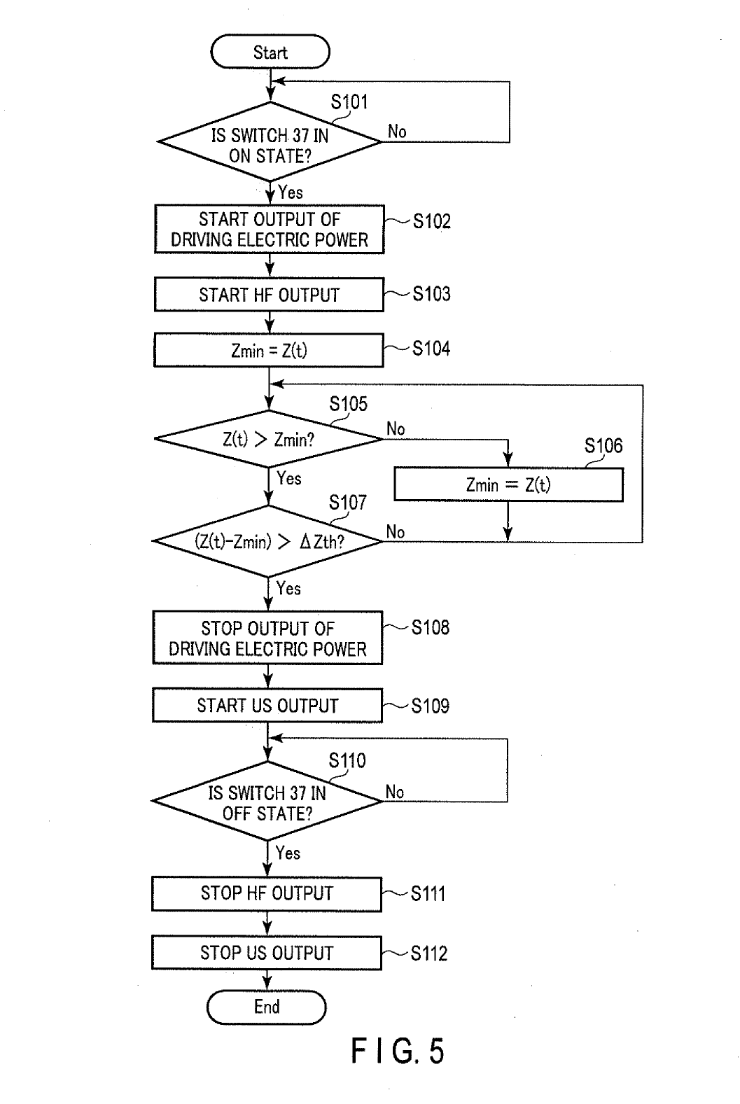

FIG. 5 is a flowchart showing a process executed by the processor 35. As shown in FIG. 5, the processor 35 determines whether or not the switch 37 is in the ON state, that is, whether or not an operation input is made through the operation button 33 (step S101). When the switch 37 is in the OFF state (No in step S101), the process returns to step S101. That is, the processor (controller) 35 waits until the operation input is made through the operation button 33 and the state of the switch 37 becomes the ON state.

When the switch 37 is in the ON state (Yes in step S101), the processor 35 starts an output of driving electric power from the driving electric power source 52 to the actuator 51 (step S102). By doing so, for example, in the example of FIG. 3, the stopper member 57 moves to the first position indicated by the broken line. Accordingly, the state of the end effector 7 becomes the first grasping state described above, and the grasping force and the grasping pressure between the grasping pieces 15 and 16 become larger than those in the state where the driving electric power is not output from the driving electric power source 52. Then, the processor 35 starts an output of electric energy from the energy output source 41 to the electrode 21 and 22, that is, an HF output (high-frequency output) (step S103). The HP output is preferably started after the movement of the stopper member 57 to the first position indicated by the broken line is completed. By doing so, the high-frequency current is applied to the grasped treatment target through the end effector 7, that is, the electrodes 21 and 22, thereby coagulating or sealing the treatment target. At this time, an output of electric energy from the energy output source 47 to the ultrasonic transducer 27, that is an US output (ultrasonic output) is not made, and an ultrasonic vibration is not applied to the treatment target.

In the present embodiment, when the output of the electric energy from the energy output source 41 is started, the processor 35 detects impedance Z of the grasped treatment target based on the output current I and the output voltage V from the energy output source 41. Here, a time t and impedance Z(t) at the time t are defined as variables. In addition, a minimum value of impedance Z from the start of the output of the electric energy from the energy output source 41 to the time t is a minimum impedance value Zmin.

In the present embodiment, when the output of the electric energy from the energy output source 41 is started, the processor 35 sets impedance Z(t) as the minimum impedance value Zmin at the time of the start of the output from the energy output source 41 or right after the start of the output from the energy output source 41 (step S104). Then, the processor 35 determines whether or not the impedance Z(t) is larger than the minimum impedance value Zmin (step S105). When the impedance Z(t) is the minimum impedance value Zmin or less (No in step S105), the processor 35 updates the minimum impedance value Zmin to the impedance Z(t) (step S106). Then, the process returns to step S105, and processes after step S105 are sequentially performed. Meanwhile, the impedance Z(t) is larger than the minimum impedance value Zmin (Yes in step S105), and the process proceeds to step S107. By the processes in steps S105 and S106, the processor 35 can detect a time at which a state where the impedance Z is gradually decreased is switched to a state where the impedance Z is gradually increased.

Then, in step S107, the processor 35 determines whether or not a difference value (Z(t)−Zmin) obtained by subtracting the minimum impedance value Zmin from the impedance Z(t) is larger than a predetermined threshold value ΔZth. When the difference value (Z(t)−Zmin) is equal to or smaller than the predetermined threshold value ΔZth (No in step S107), the process returns to step S105, and processes after step S105 are sequentially performed. When the difference value (Z(t)−Zmin) is larger than the predetermined threshold value ΔZth (Yes in step S107), the process proceeds to step S108. By the process in step S107, the processor 35 can determine whether or not an increment of the impedance from the time, at which the state where the impedance Z is gradually decreased is switched to the state where the impedance Z is gradually increased, is larger than the predetermined threshold value ΔZth. It should be noted that the predetermined threshold value ΔZth may be set by a surgeon or the like through an input section (not shown) provided in the control device 3, or may be stored in the storage medium 36. Further, the predetermined threshold value ΔZth may be fixed to a preset value. Further, the predetermined threshold value ΔZth may be set to one value of a plurality of options or may be calculated by using a function based on a change with time in the impedance Z, or the like.

In step S108, the processor 35 stops the output of the driving electric power from the driving electric power source 52 to the actuator 51. By doing so, for example, in the example of FIG. 3, the stopper member 57 moves to the second position indicated by the solid line. Accordingly, the state of the end effector 7 becomes the second grasping state described above, and the grasping force and the grasping pressure between the grasping pieces 15 and 16 become smaller than those in the first grasping state where the driving electric power is output from the driving electric power source 52. Then, the processor 35 starts an output of electric energy from the energy output source 47 to the ultrasonic transducer 27 right after the output of the driving electric power from the driving electric power source 52 is stopped (step S109). That is, a state of the output of electric energy from the energy output source 47 is switched. By doing so, an ultrasonic vibration is applied to the grasped treatment target, such that the treatment target is cut open. At this time, the output of the electric energy from the energy output source 41 to the electrodes 21 and 22 is continued, such that the high-frequency current is continuously applied to the treatment target.

Processes in steps S108 and S109 are performed in a state where the ON state of the switch 37 is maintained. Accordingly, the driving state of the actuator 51 is switched in the state where the ON state of the switch 37 is maintained, such that the state of the end effector 7 is switched from the first grasping state to the second grasping state. Then, in the state where the ON state of the switch 37 is maintained, the US output from the energy output source 47 is started. Further, the process in step S109 is performed right after the process in step S108. Accordingly, the process in step S109 is not performed until the process in step S108 is performed, and is not performed right before the process in step S108.

Then, the processor 35 determines whether or not the switch 37 is in the OFF state, that is, whether or not an operation input is released through the operation button 33 (step S110). When the switch 37 is in the ON state (No in step S110), the process returns to step S110. That is, the processor (controller) 35 continues the HF output and the US output described above and maintains the state where the output of the driving electric power is stopped until the operation input through the operation button 33 is released. Then, when the switch 37 is in the OFF state (Yes in step S110), the processor 35 stops the output of the electric energy from the energy output source 41 (step S111), and stops the output of the electric energy from the energy output source 47 (step S112). By doing so, none of the ultrasonic vibration and the high-frequency current is applied to the treatment target.

In one example, whether or not a predetermined time has elapsed from the start of the US output in step S109 may be determined, instead of the process in step S110. In this case, the processor 35 stops the HF output and the US output described above based on the fact that the predetermined time has elapsed from the start of the output of the electric energy from the energy output source 47.

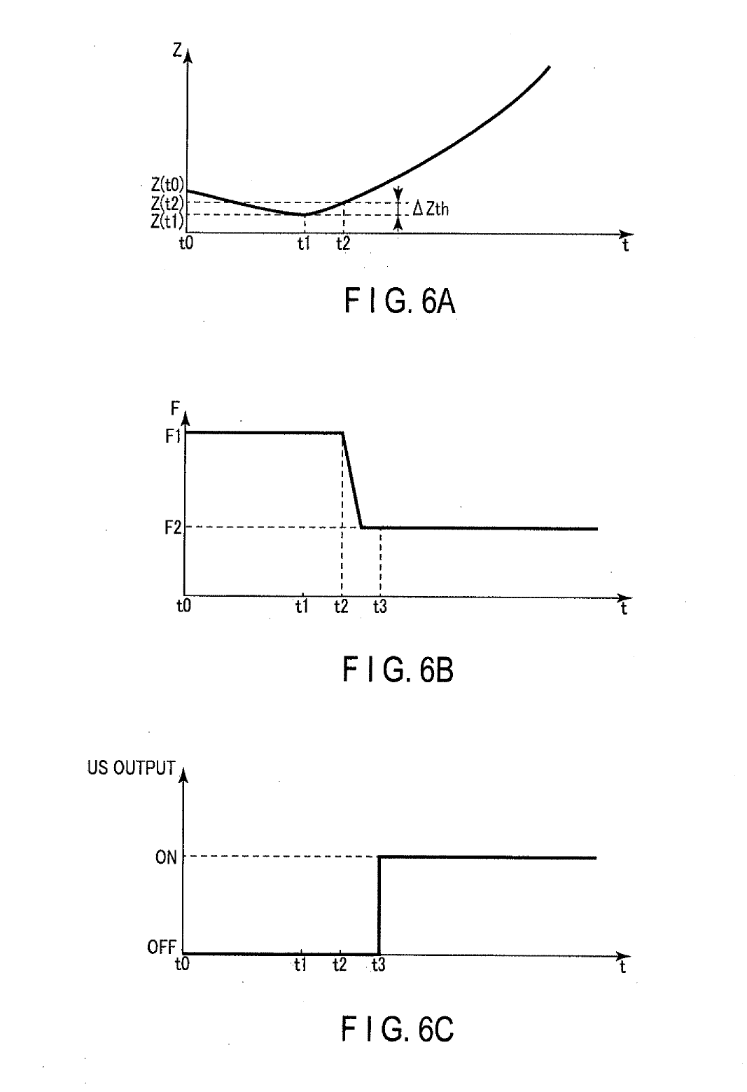

FIG. 6A shows an example of a change with time in impedance Z in the state where the treatment energy is applied to the treatment target, FIG. 6B shows a change with time in a grasping force F between the grasping pieces 15 and 16 when the impedance Z is changed as shown in FIG. 6A, and FIG. 6C shows switching with time between an ON state and an OFF state of the US output when the impedance Z is changed as shown in FIG. 6A. In FIGS. 6A to 6C, an abscissa axis represents time t. Further, an ordinate axis represents impedance Z in FIG. 6A, an ordinate axis represents a grasping force F in FIG. 6B, and an ordinate axis represents the ON state and the OFF state of the US output in FIG. 6C.

Here, when the HF output from the energy output source 41 is started and the high-frequency current starts to flow between the electrodes 21 and 22 through the grasped treatment target, the impedance Z is gradually decreased until moisture in the treatment target (in a biological tissue) evaporates by heat caused by the high-frequency current. Then, after the moisture in the treatment target evaporates, the impedance Z is gradually increased in response to an increase in a temperature of the treatment target by heat caused by the high-frequency current. For this reason, in the example shown in FIG. 6A, impedance Z(t) is gradually decreased from impedance Z(t0) at a time t0 which is a time at which the HF output is started. Then, a state of the impedance Z(t) is switched at a time t1 from a state where the impedance Z(t) is gradually decreased to a state where the impedance Z(t) is gradually increased. Therefore, the impedance Z(t) is decreased to impedance Z(t1). Then, the impedance Z(t) is gradually increased after the time t1. Then, a state where an increment from the impedance Z(t1) is equal to or smaller than the predetermined threshold value ΔZth is switched to a state where the increment from the impedance Z(t1) is larger than the predetermined threshold value ΔZth at a time t2. That is, an increment from the impedance Z(t1) to the impedance Z(t2) at the time t2 is larger than the predetermined threshold value ΔZth. In the example shown in FIG. 6A, as the high-frequency current is continuously applied as the first treatment energy, a state of the treatment target becomes a predetermined state where the moisture evaporates to some extent and the treatment target is coagulated to some extent at the time t2.

In the present embodiment, the processes shown in FIG. 5 are performed. For this reason, when the impedance Z is changed as shown in FIG. 6A, the minimum impedance value Zmin is continuously updated to the impedance Z(t) by the processes in steps S105 and S106 from the time t0, which is the time at which the HF output is started, to the time t1. Then, right after the time t1, it is determined that the impedance Z(t) is larger than the impedance Z(t1) which is the minimum impedance value Zmin by the process in step S105. Then, the impedance Z(t1) is maintained as the minimum impedance value Zmin after the time t1. Further, it is determined that the difference value (Z(t)−Zmin) is equal to or smaller than the predetermined threshold value ΔZth by the process in step S107 until the time t2. Then, it is determined that the difference value (Z(t)−Zmin) is larger than the predetermined threshold value ΔZth by the process in step S107 at the time t2 or right after the time t2. Therefore, in the example of FIG. 6A, it is determined that the state of the treatment target has become the predetermined state, where the moisture has evaporated to some extent and the treatment target is coagulated to some extent, based on the impedance Z at the time t2 or right after the time t2.

Further, since the processes shown in FIG. 5 are performed in the present embodiment, the state of the end effector 7 is switched to the first grasping state by the process in step S102 at the time at which the HF output is started or right before the HF output is started. Accordingly, when the impedance Z is changed as shown in FIG. 6A, the state of the end effector 7 is switched to the first grasping state at the time t0 or right before the time t0. Then, the end effector 7 maintains the first grasping state until the time t2. For this reason, the grasping force F between the grasping pieces 15 and 16 is a large first grasping force F1 until the time t2. Further, the grasping pressure between the grasping pieces 15 and 16 also becomes larger until the time t2.

Then, in one example of FIG. 6A, since it is determined that the difference value (Z(t)−Zmin) is larger than the predetermined threshold value ΔZth at the time t2 or right after the time t2, the process in step S108 is performed. By doing so, the state of the end effector 7 is switched from the first grasping state to the second grasping state at the time t2 or right after the time t2. Then, the end effector 7 maintains the second grasping state until, for example, the switch 37 is in the OFF state after the time t2. For this reason, the grasping force F between the grasping pieces 15 and 16 is a second grasping force F2 smaller than the first grasping force F1 after the time t2. Further, after the time t2, the grasping pressure between the grasping pieces 15 and 16 becomes smaller in comparison to the case of the first grasping state before the time t2. Here, the second grasping force F2 has a magnitude smaller by 10% to 50% than that of the first grasping force F1. Similarly, the grasping pressure in the second grasping state is smaller by 10% to 50% than the grasping pressure in the first grasping state.

Then, when the impedance Z is changed as shown in FIG. 6A, the US output from the energy output source 47 is started by the process in step S109 in response to the fact that the state of the end effector 7 has been switched to the second grasping state. By doing so, the ultrasonic vibration, which is the second treatment energy, starts to be applied to the treatment target. The output of the electric energy from the energy output source 47 is started at a time t3 after the time t2, and is started right after the state of the end effector 7 is switched to the second grasping state. Then, the US output and the HF output are continued after the time t3 until, for example, the switch 37 is in the OFF state.

As described above, in the present embodiment, the grasping force F and the grasping pressure between the grasping pieces 15 and 16 become large in a state where only the high-frequency current is applied to the treatment target. As the grasping force F and the grasping pressure become large, a treatment target sealing performance of the high-frequency current is improved. As a result, a pressure resistance value (resistance to the blood flow to the sealed region) or the like is secured for the treatment target such as the blood vessel, or the like being sealed or being cut open, such that the treatment target is appropriately sealed.

Further, in the present embodiment, the driving state of the actuator 51 is switched based on the fact that the state of the treatment target has become the predetermined state where the treatment target is coagulated to some extent, such that the grasping force F and the grasping pressure between the grasping pieces 15 and 16 are decreased. Then, the output (US output) of the electric energy from the energy output source 47 is started in response to the fact that the grasping force F and the grasping pressure has been decreased, and the ultrasonic vibration starts to be applied to the treatment target. For this reason, the grasping force F and the grasping pressure between the grasping pieces 15 and 16 become smaller in a state where the ultrasonic vibration is applied to the treatment target. As the grasping force F and the grasping pressure become smaller, deflection of the first grasping piece 15 caused by pressing of the second grasping piece 16 is suppressed, such that a load on the rod member 13 vibrating by the ultrasonic vibration is suppressed from being increased excessively. As a result, the rod member 13 vibrates appropriately, and the ultrasonic vibration is appropriately transmitted to the first grasping piece 15. Accordingly, the grasped treatment target is appropriately cut open, and the treatment target cut-open performance by the ultrasonic vibration is secured.

As described above, in the present embodiment, since the grasping force F and the grasping pressure between the grasping pieces 15 and 16 are switched as described above, the treatment target is appropriately coagulated and cut open by using the high-frequency current and the ultrasonic vibration.

### Modifications

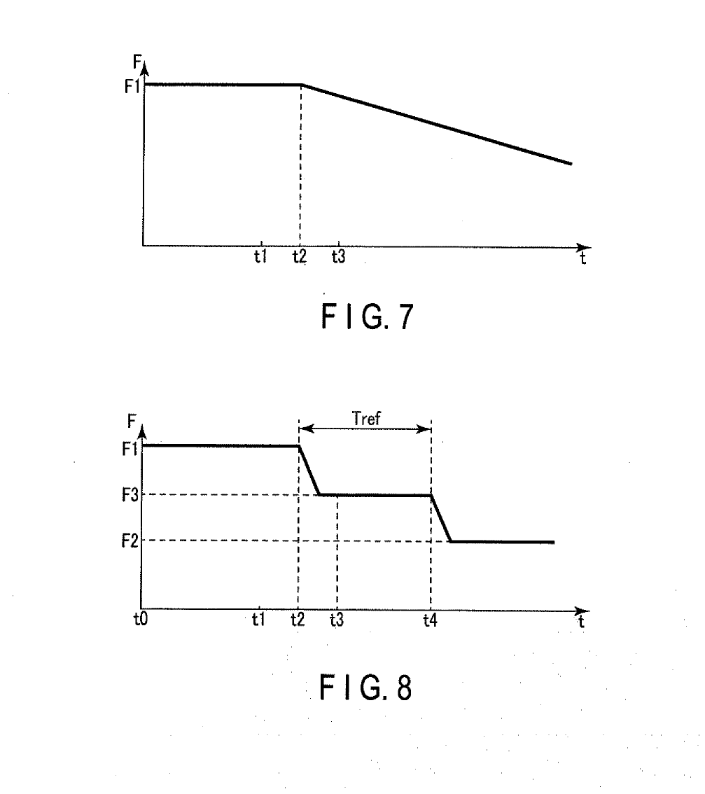

In the first embodiment, when it is determined that the state of the treatment target becomes the predetermined state, the driving state of the actuator 51 is switched, such that the grasping force between the grasping pieces 15 and 16 is switched from the first grasping force F1 to the second grasping force F2. However, it is not limited thereto. For example, as shown in FIG. 7 as one modification, when it is determined that the state of the treatment target becomes the predetermined state, the processor 35 may gradually decrease the grasping force from the first grasping force F1. In this case, the grasping pressure between the grasping pieces 15 and 16 also is also gradually decreased from that before it is determined that the state of the treatment target has become the predetermined state, simultaneously with the decrease in the grasping force. In the present modification, the processor 35 gradually decreases the grasping force and the grasping pressure by gradually decreasing a magnitude of the driving electric power supplied to the actuator 51 from that before it is determined that the state of the treatment target has become the predetermined state. It should be noted that FIG. 7 shows a change with time in the grasping force F when the impedance Z is changed as shown in FIG. 6A, an abscissa axis represents time t, and an ordinate axis represents the grasping force F.

Also in the present modification, the driving state of the actuator 51 is switched based on the fact that it is determined that the state of the treatment target has become the predetermined state, such that the grasping state of the end effector 7 is switched. Then, the grasping force and the grasping pressure between the grasping pieces 15 and 16 become smaller in the second grasping state after it is determined that the state of the treatment target has become the predetermined state, than in the first grasping state before it is determined that the state of the treatment target has become the predetermined state.

Further, in another modification shown in FIG. 8, when it is determined that the state of the treatment target has become the predetermined state, the processor 35 decreases the grasping force F to a third grasping force F3 which is smaller than the first grasping force F1 and larger than the second grasping force F2. Then, the processor 35 further decreases the grasping force to the second grasping force F2 at a point in time when a predetermined time Tref has elapsed from a time at which switching to the third grasping force F3 is made. In this case, the grasping pressure between the grasping pieces 15 and 16 is also decreased from that before it is determined that the state of the treatment target has become the predetermined state, simultaneously with the decrease in the grasping force to the third grasping force F3. Then, the grasping pressure is further decreased, simultaneously with the decrease in the grasping force to the second grasping force F2. In the present modification, the processor 35 decreases the grasping force to the third grasping force F3 by decreasing a magnitude of the driving electric power supplied to the actuator 51 from that before it is determined that the state of the treatment target has become the predetermined state. Then, the processor 35 decreases the grasping force to the second grasping force F2 by stopping the output of the driving electric power, or by further decreasing the magnitude of the driving electric power from that in a state of the second grasping force F2.

It should be noted that FIG. 8 shows a change with time in the grasping force F when the impedance Z is changed as shown in FIG. 6A, an abscissa axis represents time t, and an ordinate axis represents the grasping force F. In FIG. 8, the grasping force F is decreased to the third grasping force F3 at the time t2 or right after the time t2. Then, the grasping force F is further decreased to the second grasping force F2 at a time t4 when a reference time Tref has elapsed from the time t2, or right after the time t4.

Also in the present modification, the driving state of the actuator 51 is changed based on the fact that it is determined that the state of the treatment target has become the predetermined state, such that the grasping state of the end effector 7 is switched. Then, the grasping force and the grasping pressure between the grasping pieces 15 and 16 becomes smaller in the second grasping state after it is determined that the state of the treatment target has become the predetermined state, than in the first grasping state before it is determined that the state of the treatment target has become the predetermined state.

Further, in another modification, an output current I′ and an output voltage V′ from the energy output source 47 are detected in a state where the electric energy is output from the energy output source 47 to the ultrasonic transducer 27. In this case, the processor 35 detects impedance Z′ of the ultrasonic transducer 27 based on the output current I′ and the output voltage V′. Here, the impedance Z′ of the ultrasonic transducer 27 is changed depending on the load to the rod member 13. The processor 35 controls the driving of the actuator 51 based on the impedance Z′ and adjusts the grasping force and the grasping pressure between the grasping pieces 15 and 16 in a state where the electric energy is output from the energy output source 47 to the ultrasonic transducer 27. However, even in the present modification, the grasping force and the grasping pressure between the grasping pieces 15 and 16 become smaller in the second grasping state after it is determined that the state of the treatment target has become the predetermined state, than in the first grasping state before it is determined that the state of the treatment target has become the predetermined state. Therefore, the processor 35 adjusts the grasping force within a range smaller than the grasping force in the first grasping state based on the impedance Z′. Then, the processor 35 adjusts the grasping pressure within a range smaller than the grasping pressure in the first grasping state based on the impedance Z′.

Further, in the embodiment and the like described above, whether or not the state of the treatment target has become the predetermined state is determined by the processes in steps S104 to S107 of FIG. 5. That is, it is determined that the state of the treatment target has become the predetermined state based on the fact that switching from the state where the impedance Z(t) of the treatment target is gradually decreased to the state where the impedance Z(t) of the treatment target is gradually increased is made and the fact that the increment of the impedance Z(t) from the time at which a gradual increase in the impedance Z(t) is started has become larger than the predetermined threshold value ΔZth. However, in one modification, the processor 35 may determine that the state of the treatment target has become the predetermined state where the moisture has evaporated to some extent and the treatment target is coagulated to some extent based on the fact that the impedance Z(t) has become larger than a predetermined threshold value Zth1. In this case, the predetermined threshold value Zth1 is set to a value equal to or larger than the impedance Z(t0) at the time t0 which is the time at which the output (HF output) of the electric energy from the energy output source 41 is started. Further, in another modification, the processor 35 determines that the state of the treatment target has become the predetermined state where the moisture has evaporated to some extent and the treatment target is coagulated to some extent based on the fact that the impedance Z(t) has become smaller than a predetermined threshold value Zth2 and then has become larger than the predetermined threshold value Zth2 again. In this case, the predetermined threshold value Zth2 is set to a value equal to or smaller than the impedance Z(t0) at the time t0 which is the time at which the HF output is started.

Further, in one modified example, the processor 35 may determine whether or not the state of the treatment target has become the predetermined state by using the phase difference θ between the output current I and the output voltage V from the energy output source 41 in place of the impedance Z. In a state in which the HF output from the energy output source 41 is started and the high-frequency current flows to the treatment target, a change with time in the phase difference θ shows the same tendency as that of the change with time in the impedance Z. That is, when the high-frequency current starts to be applied to the treatment target, the phase difference θ is gradually decreased to close to zero until the moisture in the treatment target (in a biological tissue) evaporates by heat caused by the high-frequency current. Then, the phase difference θ is gradually increased after the moisture in the treatment target evaporates.

Accordingly, in one example, the processor 35 determines whether or not the state of the treatment target has become the predetermined state based on the fact that switching from the state where a phase difference θ(t) is gradually decreased to the state where the phase difference θ(t) of the treatment target is gradually increased is made and fact that an increment of the phase difference θ(t) from the time at which a gradual increase in the phase difference θ(t) is started has become larger than a predetermined threshold value Δθth. In another example, the processor 35 may determine that the state of the treatment target has become the predetermined state based on the fact that the phase difference θ(t) has become larger than a predetermined threshold value θth1. In this case, the predetermined threshold value θth1 is set to a value equal to or larger than a phase difference θ(t0) at the time t0 which is the time at which the output (HF output) of the electric energy from the energy output source 41 is started. Further in still another example, the processor 35 determines that the state of the treatment target has become the predetermined state based on the fact that the phase difference θ(t) has become smaller than a predetermined threshold value θth2 and the has become larger than the predetermined threshold value θth2 again. In this case, the predetermined threshold value θth2 is set to a value equal to or smaller than the phase difference θ(t0) at the time t0 which is the time at which the HF output is started.

Further, in one modification, the processor 35 may determine whether or not the state of the treatment target has become the predetermined state based on the duration time T for which the HF output is continued from the start of the HF output. In this case, the processor 35 determines that the treatment target has become the predetermined state based on the fact that the duration time T is equal to or longer than a predetermined time Tth.

Further, in the embodiment and the like described above, the high-frequency current is the first treatment energy coagulating the treatment target, but it is not limited thereto. In one modification, a heating element such as a heater or the like may be provided in the end effector 7, and the grasped treatment target may be coagulated by heat generated from the heating element. In this case, an energy output source outputting direct current (DC) electric power or AC electric power as electric energy to the heating element is provided in the control device 3. The energy output source converts electric power from a battery power source or a power receptacle into electric energy supplied to the heating element. Then, as the electric energy is supplied from the energy output source to the heating element, heat is generated from the heating element, and the generated heat is applied as first treatment energy to the treatment target. At this time, the processor 35 controls the output of the electric energy to the heating element so that a temperature of the heating element is equal to or lower than 100° C. That is, the temperature of the heating element is controlled so that the treatment target is not cut open by the heat generated from the heating element.

In the embodiment and the like described above, the processor (35) switches the driving state of the actuator (51) based on the fact that the state of the treatment target has become the predetermined state in the state where the electric energy is output from the energy output source (41). As the driving state of the actuator (51) is switched, the state of the end effector (7) is switched from the first grasping state to the second grasping state where the grasping force and the grasping pressure between the first grasping piece (15) and the second grasping piece (16) are different from those of the first grasping state.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An energy treatment instrument used with a control device, the energy treatment instrument comprising:
an end effector which includes a first grasping piece, and a second grasping piece being openable and closable relative to the first grasping piece, the end effector being configured to grasp a treatment target between the first grasping piece and the second grasping piece, and treatment energy being configured to be applied to the grasped treatment target;
an actuator which is configured to be driven in accordance with a control by a processor of the control device, a driving state of the actuator being configured to be switched based on a fact that a state of the treatment target has become a predetermined state when the treatment energy is applied to the grasped treatment target, so that a state of the end effector is switched from a first grasping state to a second grasping state, and the actuator being configured to make a grasping force and a grasping pressure between the first grasping piece and the second grasping piece in the second grasping state different from those in the first grasping state;
a housing which is hand-holdable; and
a handle which is configured to open or close relative to the housing so as to open or close the first grasping piece and the second grasping piece relative to each other,
wherein a stroke of the handle in a closing motion is configured to be changed in accordance with a switch of the driving state of the actuator, so that the state of the end effector is switched from the first grasping state to the second grasping state.

2. The energy treatment instrument according to claim 1, wherein
the actuator is configured to make the grasping force and the grasping pressure between the first grasping piece and the second grasping piece in the second grasping state smaller than those in the first grasping state.

3. The energy treatment instrument according to claim 1, further comprising:
a switch which is configured to make the treatment energy be applied to the grasping treatment target, when being in an ON state,
wherein the driving state of the actuator is configured to switch when the ON state of the switch is maintained, so that the state of the end effector is switched from the first grasping state to the second grasping state.

4. The energy treatment instrument according to claim 1, wherein
first treatment energy, which is one of the treatment energy and coagulates the grasped treatment target, is applied through the end effector in the first grasping state, and
second treatment energy, which is one of the treatment energy and cuts open the grasped treatment target, starts to be applied through the end effector based on a fact that the state of the end effector has been switched from the first grasping state to the second grasping state.

5. The energy treatment instrument according to claim 4, wherein
the first grasping piece includes a first electrode,
the second grasping piece includes a second electrode, and
electric energy is supplied to the first electrode and the second electrode, so that a high-frequency current flows as the first treatment energy between the first electrode and the second electrode through the grasped treatment target in the end effector.

6. The energy treatment instrument according to claim 4, further comprising:
an ultrasonic transducer which is configured to generate an ultrasonic vibration by a supply of electric energy and configured to transmit the generated ultrasonic vibration to the first grasping piece,
wherein the ultrasonic vibration transmitted to the first grasping piece is applied as the second treatment energy to the grasped treatment target.

7. The energy treatment instrument according to claim 1, further comprising:
an elastic member, a contraction state of the elastic member being configured to be changed so that the grasping force and the grasping pressure between the first grasping piece and the second grasping piece are changed,
wherein the contraction state of the elastic member is configured to be changed in accordance with the switch of the driving state of the actuator, so that the state of the end effector is switched from the first grasping state to the second grasping state.

8. A control device used with an energy treatment instrument and an actuator, the energy treatment instrument including a first grasping piece, a second grasping piece, a hand-holdable housing, and a handle, the second grasping piece being openable and closable relative to the first grasping piece, the handle being configured to open or close relative to the housing so as to open or close the first grasping piece and the second grasping piece relative to each other, an end effector configured to grasp a treatment target between the first grasping piece and the second grasping piece being provide in the energy treatment instrument, the control device comprising:
an energy output source configured to output electric energy, the energy output source being configured to supply the output electric energy to the energy treatment instrument so that treatment energy is applied to the grasped treatment target through the end effector; and
a processor which is configured to control an output of the electric energy from the energy output source and control a driving of the actuator, the processor being configured to switch a driving state of the actuator based on a fact that a state of the treatment target has become a predetermined state when the electric energy is output from the energy output source, so that a stroke of the handle in a closing motion is changed, a state of the end effector being configured to be switched from a first grasping state to a second grasping state in accordance with a change of the stroke of the handle, the actuator being configured to make a grasping force and a grasping pressure between the first grasping piece and the second grasping piece in the second grasping state different from those in the first grasping state.

9. The control device according to claim 8, wherein
the processor is configured to control the driving of the actuator so that the actuator makes the grasping force and the grasping pressure between the first grasping piece and the second grasping piece in the second grasping state smaller than those in the first grasping state.

10. The control device according to claim 8, wherein
the energy output source is configured to supply the electric energy to the first grasping piece and the second grasping piece so that a high-frequency current flow as the treatment energy through the treatment target grasped between the first grasping piece and the second grasping piece, and
the processor is configured to detect at least one of impedance of the treatment target and a phase difference between an output current and an output voltage in an output from the energy output source to the first grasping piece and the second grasping piece, and configured to determine whether or not the state of the treatment target has become the predetermined state based on the detected impedance and/or phase difference.

11. The control device according to claim 8, wherein
the processor is configured to detect a duration time for which an output of the electric energy from the energy output source is continued from a start of the output, and configured to determine whether or not the state of the treatment target has become the predetermined state based on the duration time.

12. The control device according to claim 8, wherein
the processor is configured to control the output of the electric energy from the energy output source in the first grasping state of the end effector so that first treatment energy coagulating the grasped treatment target is applied to the treatment target through the end effector, and
the processor is configured to switch an output state of the electric energy from the energy output source in response to a fact that the state of the end effector has been switched from the first grasping state to the second grasping state, so that second treatment energy cutting open the grasped treatment target start is applied to the treatment target.

13. The control device according to claim 8, wherein
the processor is configured to make the energy output source output the electric energy to the energy treatment instrument in an ON state of a switch, the switch being used with the control device, and
the processor is configured to switch the driving state of the actuator when the ON state of the switch is maintained, so that the state of the end effector is switched from the first grasping state to the second grasping state.

14. A treatment system, comprising:
the control device according to claim 8;
the energy treatment instrument which includes the first grasping piece and the second grasping piece; and
the actuator configured to adjust the grasping force and the grasping pressure between the first grasping piece and the second grasping piece.
